(19) United States
(12) Patent Application Publication
(10) Pub. No.: US 2025/0172497 A1
(43) Pub. Date: May 29, 2025

(54) OPTICAL MEASURING DEVICE FOR DETERMINING A MEASURED VARIABLE IN WATER

(71) Applicant: Endress+Hauser Conducta GmbH+Co. KG, Gerlingen (DE)

(72) Inventors: Andreas Bayer, Gerlingen (DE); Thilo Krätschmer, Gerlingen (DE)

(21) Appl. No.: 18/963,030

(22) Filed: Nov. 27, 2024

(30) Foreign Application Priority Data
Nov. 27, 2023 (DE) 10 2023 133 005.5

(51) Int. Cl. G01N 21/64; G01N 33/18

(52) U.S. Cl. CPC G01N 21/645; G01N 21/6486; G01N 33/1833; G01N 33/1886

(57) ABSTRACT

An optical measuring arrangement for determining a measured variable in a medium includes: at least one light source which transmits excitation light into the water in the direction of the water surface, wherein the excitation light is converted into fluorescent light in the water and on the water surface; at least one photodiode which receives the fluorescent light from the water and from the water surface and converts the fluorescent light into an electrical signal; and a data processing unit which determines the measured variable from the electrical signal. A corresponding method for determining the measured variable in the medium uses the optical measuring arrangement.

### CROSS-REFERENCE TO RELATED APPLICATION

The present application is related to and claims the priority benefit of German Patent Application No. 10 2023 133 005.5, filed Nov. 27, 2023, the entire contents of which are incorporated herein by reference.

### TECHNICAL FIELD

The present disclosure relates to an optical measuring arrangement for determining a measured variable in water, in particular for determining an oil-in-water content.

### BACKGROUND

Not only water in technical systems (e.g., scrubbers for exhaust gas cleaning on ships) but also natural surface water can be contaminated with oil or plastic particles, among other things. A well-known way to detect such contamination is fluorescence measurement. In particular, UV fluorescence can be used to quantitatively determine the content of polyaromatic cyclic hydrocarbons, for example. Depending on the pretreatment, the presence of solubilizers or suspended particles and depending on turbulence in the liquid, the medium to be measured, in particular oil, is either distributed throughout the volume of the liquid or floats at least partially on the surface.

However, available fluorescence sensors either measure in the liquid volume or are directed onto water surfaces from above. For example, the fluorescence sensor “CFS51” from the Endress+Hauser Group is suitable for ship exhaust gas cleaning and is installed in a flow-through fitting or is immersed in a basin, channel, river or the sea. Other fluorescence sensors are optionally available with a float for measuring either parallel under the water surface or directly on the water surface.

Current measurement principles therefore detect the analyte either in the volume or on the surface, but not both at the same time. For instance, conventional immersion sensors detect fluorescence in the volume but cannot detect oil droplets floating on the surface. Sensors for surface measurement are less sensitive to substances that are distributed in the volume.

### SUMMARY

The present disclosure is based on the object of detecting fluorescent analytes in water more reliably and with greater sensitivity.

The object is achieved by an optical measuring arrangement for determining a measured variable in water, in particular for determining the oil-in-water content, the content of microplastics or the content of algae, the arrangement comprising: at least one light source, which transmits excitation light into the water in the direction of the water surface, wherein the excitation light is converted into fluorescent light in the water and on the water surface; at least one photodiode, which receives the fluorescent light from the water and from the water surface and converts said fluorescent light into an electrical signal; and a data processing unit, which determines the measured variable from the electrical signal.

The disclosed arrangement results in a fluorescence sensor which is directed from bottom to top and which can be arranged directly below the water surface in order to detect oil, microplastics or algae in the liquid volume and simultaneously on the surface. In this case, total reflection at the liquid surface can be utilized to improve the sensitivity of the measurement system. The disclosed arrangement thus describes the possibility of simultaneously detecting fluorescent substances in the liquid volume and on the surface.

In at least one embodiment, the light source, the photodiode and the data processing unit are arranged in a housing and the light source and photodiode are in optical communication with the water via a common or individual optical window in each case.

In at least one embodiment, the light source and the photodiode are arranged below the water surface.

In at least one embodiment, the light source and the photodiode are arranged in the water.

In at least one embodiment, the angle of incidence of the excitation light into the water is such that total reflection occurs at the water surface.

In at least one embodiment, the distance from the light source and photodiode to the water surface is variable.

In at least one embodiment, the angle of incidence of the excitation light and/or the detection angle of the fluorescent light is variable.

In at least one embodiment, the arrangement comprises one or more floating bodies which hold the measuring arrangement at a defined distance from the water surface, wherein the floating body is arranged outside the measuring arrangement.

In at least one embodiment, the measuring arrangement comprises at least one swim bladder which holds the measuring arrangement at a defined distance from the water surface, wherein the swim bladder is arranged in the measuring arrangement.

In at least one embodiment, the measuring arrangement comprises a tub with an inlet and an outlet for the water, wherein the light source and the photodiode are arranged in the tub.

In at least one embodiment, the measuring arrangement comprises a tub with an inlet and an outlet for the water, wherein the light source and the photodiode are arranged outside the tub

The object is further achieved by a method comprising the steps of: transmitting excitation light into the water in the direction of the water surface, wherein the excitation light is converted into fluorescent light in the water and at the water surface; receiving the fluorescent light from the water and from the water surface; converting the fluorescent light into an electrical signal; and determining the measured variable from the electrical signal.

The object is furthermore achieved by using a measuring arrangement as described above to determine the oil-in-water content.

The object is furthermore achieved by using a measuring arrangement as described above to determine the content of microplastics.

The object is furthermore achieved by using a measuring arrangement as described above to determine the content of algae.

### BRIEF DESCRIPTION OF THE DRAWINGS

The present disclosure is explained in more detail with reference to the following figures.

FIG. 1 shows an overview of the measuring arrangement according to the present disclosure.

FIG. 2 shows an embodiment of the measuring arrangement according to the present disclosure.

FIG. 3 shows an embodiment of the measuring arrangement according to the present disclosure.

FIGS. 4a and 4b show different embodiments of the measuring arrangement according to the present disclosure.

In the figures, the same features are labeled with the same reference signs.

### DETAILED DESCRIPTION

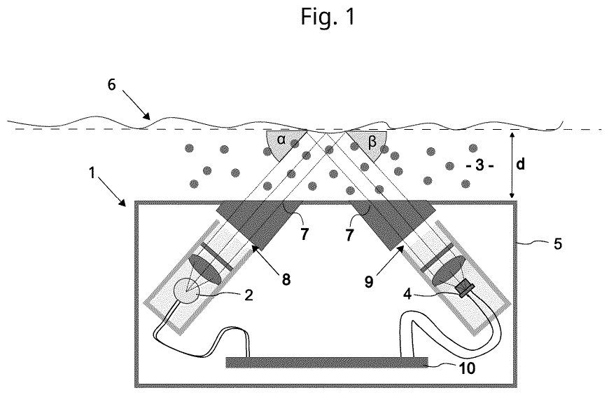

The measuring arrangement according to the present disclosure in its entirety has reference sign 1 as shown in FIG. 1.

The measuring arrangement 1 comprises a light source 2, a photodiode 4 (e.g., a detector) and a data processing unit 10. The measuring arrangement 1 is interchangeably referred to as a “sensor” herein.

The sensor may be a fluorescence sensor. In order to measure fluorescence, the medium (e.g., water) is generally irradiated with a short-wavelength excitation light 8, and the longer-wavelength fluorescent light produced by the medium is detected.

The light source 2 radiates excitation light 8 into the medium 3 to be measured, e.g., water, wherein the excitation light 8 is converted by the medium 3 into fluorescent light 9. The fluorescent light 9 is received by the photodiode 4 and converted into an electrical signal, for example, as an intensity, decay curve or phase.

The light source 2 is, for example, a UV light source which emits light with a wavelength of 200-400 nm. The light source 2 is designed as a UV flash lamp, for example. The light source 2 can also be configured as an LED. The UV flash lamp emits in the spectral range from UV to IR. Depending on the application, the light source 2 is selected and designed accordingly. For an application determining the algae content, the light source 2 may emit in the UV/VIS range (200-800 nm). The arrangement may also include other optical components in the beam path after the light source 2, such as a filter that transmits only the desired wavelengths of the fluorescence emission of the analyte to be measured, or one or more lenses. The flash lamp then emits, together with the filter, only the desired excitation wavelength. Corresponding components are also arranged on the receiver side at the photodiode 4.

The light source 2 and the photodiode 4 are connected to a data processing unit 10 (such as a microcontroller), which uses the electrical signal to determine the measured variable to be determined, such as the oil-in-water content, the proportion of microplastics in the water, or the algae content, for example. The measured variable is determined using a calibration model, which determines the concentration, for example, from the fluorescence intensity.

The light source 2, the photodiode 4 and the data processing unit 10 are arranged in a common housing 5, wherein the light source 2 and the photodiode 4 are in optical communication with the medium 3 via a common or separate optical window 7 in each case.

In order to be simultaneously sensitive to the analyte in the volume of medium 3 and at the surface 6 of the medium, both the excitation light 8 and the fluorescence light 9 are sent and received, respectively, at a certain angle α or β from below against the medium surface 6. Depending on the exact arrangement, the ratio between the sensitivity at the surface and in the volume can be adjusted. The light source 2 thus transmits through the medium 3 towards the medium surface 6. The excitation light 8 is converted into fluorescent light 9 not only in the medium 3 but also on its surface 6. The light source 2 emits excitation light 8 substantially counter to the force of gravity (although this, of course, has no influence). “Bottom” in the sense of this document is thus below medium surface in the volume, while “top” is at the surface.

The light source 2 and the photodiode 4 are thus arranged below the medium surface 6.

FIGS. 1-3 and 4b show embodiments in which the light source 2 and the photodiode 4 (together with the data processing unit 10 and housing 5) are arranged in the medium 3.

Since the fluorescent light is generally emitted in all spatial directions, the light paths of excitation light 8 and fluorescent light 9 can in principle be at any angle to one another.

If the angle a of excitation and/or the angle β of detection is selected to be appropriately small, the total reflection at the medium surface 6 results in significantly more volume being excited or detected, which significantly increases the sensitivity of the system. The exact value of the critical angle at which total reflection occurs depends on the wavelength of the excitation and fluorescence light. Broadly, the critical angle is approximately 42° in the UV range.

In at least one embodiment of the measuring arrangement 1, the distance d from the light source 2 and photodiode 4 to the medium surface 6 is adjustable. The optimal distance depends on various parameters, such as the size of the measuring device and the angles of the beam path. In one embodiment, the distance d varies within a centimeter range, for example, from 1-10 cm. In one embodiment, the distance d varies from a few millimeters to a few centimeters, approximately 10 cm. A distance of up to one meter is possible.

The measuring arrangement 1 can also be equipped with an (automatically) movable detector to determine the distribution of the analyte between the surface and volume. Likewise, the angle of incidence α or the angle of reflection β can be adjustable.

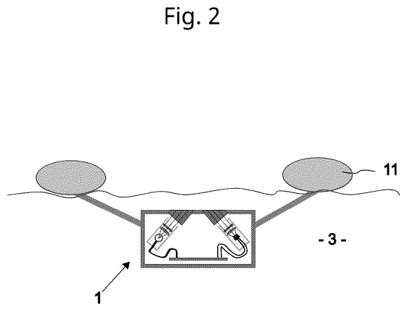

In FIGS. 2-4, only differences from or additions to FIG. 1 are marked with reference signs.

FIG. 2 shows an embodiment with two floating bodies 11, which are arranged outside the measuring arrangement 1. It is also possible to use just one. The floating body 11 is connected to the measuring arrangement 1 by lines (e.g., webs, cables, cords) and keeps the latter at a defined distance under water.

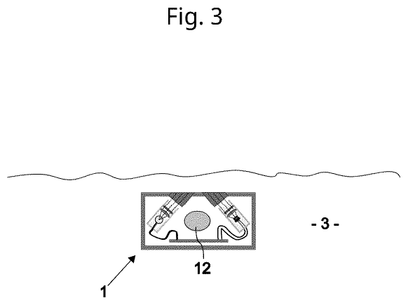

FIG. 3 shows an embodiment with a swim bladder 12, which is arranged in the housing 5. The swim bladder 12 also holds the arrangement 1 at a defined distance below the water surface 6. In one embodiment, the swim bladder 12 can be filled with air or water such that the arrangement 1 can be moved.

In both cases, continuous movability of the measuring arrangement 1 relative to the surface 6 ensures that a depth profile of the analyte concentration can be determined.

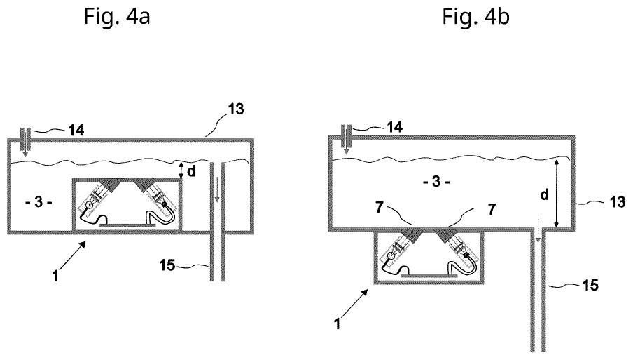

FIGS. 4a and 4b show an embodiment having a tub 13 with an inlet 14 and an outlet 15 for the medium 3. Here too, the arrangement measures from bottom to top in the direction of the medium surface 6. The distance d from the arrangement 1 to the surface can be adjusted by the inlet 14. FIG. 4a shows an embodiment in which the light source 2 and the photodiode 4 (in the housing 5) are arranged in the tub 13. FIG. 4b shows an embodiment in which these are arranged outside the tub 13. The optical window(s) 7 is/are then part of the tub 13.

In at least one embodiment, the measuring arrangement 1 is attached to a cord, rope, cable or the like and can be manually or automatically raised and lowered in the medium 3 in order to change the distance to the water surface 6.

We claim:

1. An optical measuring arrangement for determining a measured variable in water, the measuring arrangement comprising:
a light source configured to emit excitation light into the water in a direction of a surface of the water, wherein the excitation light is converted into fluorescent light in the water and on the water surface;
a photodiode configured to receive the fluorescent light from the water and from the water surface and to convert the fluorescent light into an electrical signal; and
a data processing unit configured to determine a value of the measured variable from the electrical signal.

2. The measuring arrangement according to claim 1, wherein the light source, photodiode and data processing unit are arranged in a housing, and the light source and photodiode are in optical communication with the water via a common or individual optical window in each case.

3. The measuring arrangement according to claim 1, wherein the light source and the photodiode are arranged below the water surface.

4. The measuring arrangement according to claim 1, wherein the light source and the photodiode are arranged in the water.

5. The measuring arrangement according to claim 1, wherein an angle of incidence of the excitation light into the water is such that total reflection occurs at the water surface.

6. The measuring arrangement according to claim 1, wherein a distance from the light source and photodiode to the water surface is adjustable.

7. The measuring arrangement according to claim 1, wherein an angle of incidence of the excitation light and/or a detection angle of the fluorescent light is adjustable.

8. The measuring arrangement according to claim 1, further comprising one or more floating bodies adapted to hold the measuring arrangement at a defined distance from the water surface, wherein the floating body is disposed outside the measuring arrangement.

9. The measuring arrangement according to claim 1, further comprising at least one swim bladder adapted to hold the measuring arrangement at a defined distance from the water surface, wherein the swim bladder is disposed in the measuring arrangement.

10. The measuring arrangement according to claim 1, further comprising a tub including an inlet and an outlet for the water, wherein the light source and the photodiode are arranged in the tub.

11. The measuring arrangement according to claim 1, further comprising a tub including an inlet and an outlet for the water, wherein the light source and the photodiode are arranged outside the tub.

12. The measuring arrangement according to claim 1, wherein the measure variable is an oil-in-water content, a content of microplastics, or a content of algae in the water.

13. The measuring arrangement according to claim 1, comprising more than one light source and/or more than one photodiode.

14. A method for determining a measured variable in water, the method comprising:
transmitting excitation light into the water in a direction of a surface of the water, wherein the excitation light is converted into fluorescent light in the water and at the water surface;
receiving the fluorescent light from the water and from the water surface;
converting the fluorescent light into an electrical signal; and
determining the measured variable based on the electrical signal.

15. The measuring arrangement according to claim 14, wherein the measure variable is an oil-in-water content, a content of microplastics, or a content of algae in the water.

16. A method for determining an oil-in-medium content, a content of microplastics, or a content of algae in a medium, the method comprising:
transmitting excitation light into the medium in a direction of a surface of the medium, wherein the excitation light is converted into fluorescent light in the medium and at the medium surface;
receiving the fluorescent light from the medium and from the medium surface;
converting the fluorescent light into an electrical signal; and
determining the oil-in-medium content, the content of microplastics, and/or the content of algae in the medium based on the electrical signal.

17. The measuring arrangement according to claim 16, wherein the medium is water, and wherein the oil-in-medium content is an oil-in-water content.
